(19) United States
(12) Patent Application Publication
(10) Pub. No.: US 2015/0290174 A1
(43) Pub. Date: Oct. 15, 2015

(54) PHARMACEUTICAL FORMULATIONS AND METHOD OF USING THE SAME FOR ALLEVIATING SYMPTOMS OF HANGOVER, STOMACH FLU OR MIGRAINE

(71) Applicant: Resuscitate MOE LLC, Phoenix, AZ (US)

(72) Inventor: Michael Wilmink, Phoenix, AZ (US)

(21) Appl. No.: 14/683,875

(22) Filed: Apr. 10, 2015

Related U.S. Application Data
(60) Provisional application No. 61/978,541, filed on Apr. 11, 2014.

(51) Int. Cl. A61K 31/4178; A61K 9/48; A61K 31/426; A61K 31/167; A61K 31/192

(52) U.S. Cl. CPC A61K 31/4178; A61K 31/167; A61K 31/192; A61K 31/426; A61K 9/4891

(57) ABSTRACT

Described herein are pharmaceutical formulations and dosage forms that include an analgesic agent, a nonsteroidal anti-inflammatory agent, a gastric acid production suppressant, and an anti-nauseant, which are capable of alleviating the symptoms of hangovers, stomach flu or migraine.

### CROSS-REFERENCE TO RELATED APPLICATIONS

This application claims the benefit under 35 U.S.C. §119(e) of U.S. Provisional Patent Application No. 61/978,541, filed Apr. 11, 2014, which application is incorporated herein by reference in its entirety.

### BACKGROUND

1. Description of the Related Art

This disclosure relates to pharmaceutical formulations and dosage forms for alleviating symptoms of hangover, stomach flu or migraine.

2. Description of the Related Art

Hangover is colloquially described as a feeling ranging from unpleasantness to general misery by an individual that has overindulged in consumption of alcohol. Although the pathology of alcohol hangover is not well understood, it can be characterized as a collection of physical and mental symptoms. Physical symptoms of a hangover include fatigue, headache, nausea, gastro-intestinal irritations (e.g., vomiting and diarrhea), increased sensitivity to light and sound, muscle aches, and thirst. Mental symptoms include dizziness, vertigo, cognitive and mood disturbances such as depression, anxiety, irritability, and decreased ability to concentrate.

Dehydration and metabolic abnormalities (e.g., electrolyte imbalance, metabolic acidosis) are the two most common physiological markers of hangovers. Certain symptoms, such as vomiting and diarrhea, may exacerbate existing symptoms of hangovers due to the further loss of water and electrolytes.

Hangover is not an immediate reaction to alcohol consumption. Instead, it occurs after the alcohol and its metabolites have begun to be eliminated from the body. The breakdown products of alcohol result in the production of lactic acid and the release of cytokines throughout the body.

Hangover symptoms can be sufficiently acute that urgent care is required to rehydrate and to correct the electrolyte imbalance. Such urgent care is generally carried out in an emergency room or hospital setting and includes treatment by intravenous fluids and/or intravenous medications.

Stomach flu presents a similar set of symptoms as hangover, despite having an entirely different pathology. Patients generally experience dehydration and metabolic alkalosis from frequent vomiting and diarrhea. Often urgent care is also required to address these symptoms.

Migraine also shares a significant number of symptoms with hangover and stomach flu, including, headache, nausea, vomiting and increased sensitivity to light, sound and smell.

There is a need to address the symptoms of hangovers, stomach flu or migraine and to provide relief such that urgent care may be obviated.

### BRIEF SUMMARY

Disclosed herein are various embodiments directed to pharmaceutical formulations, dosage forms and treatments that are capable of alleviating and potentially decreasing the severity of the symptoms of hangovers, stomach flu or migraine such that a patient suffering from the same will not require urgent care.

One embodiment provides a pharmaceutical composition comprising: an analgesic agent, a nonsteroidal anti-inflammatory agent, a gastric acid production suppressant, and an anti-nauseant, wherein the analgesic agent, the nonsteroidal anti-inflammatory agent, the gastric acid production suppressant, and the anti-nauseant are, respectively, in sufficient amounts to alleviate one or more symptoms of hangover, stomach flu or migraine.

Another embodiment provides a unit dosage form for alleviating one or more symptoms of hangover, stomach flu or migraine, the unit dosage form comprising: 300-650 mg of an analgesic agent: 50-800 mg of a nonsteroidal anti-inflammatory agent; 30-80 mg of a gastric acid production suppressant; and 5-25 mg of an anti-nauseant. Additional ingredients such as vitamins, magnesium salt may be added as supplements.

Yet another embodiment provides a method of alleviating one or more symptoms of hangover, stomach flu or migraine in a patient in need thereof comprising: orally administering to the patient a single unit dosage form as describe herein, wherein the symptoms include pain, nausea, and GI irritations, and wherein the patient does not require urgent care.

A further embodiment provides a method of alleviating one or more symptoms of hangover, or stomach flu or migraine in a patient in need thereof comprising orally administering to the patient, sequentially,

(1) a single unit dosage form of an anti-nauseant, followed by

(2) a single unit dosage form of an gastric acid production suppressant, followed by

(3) a single unit dosage form of an analgesic, and followed by

(4) a single unit dosage form of a nonsteroidal anti-inflammatory agent, wherein the symptoms include pain, nausea, and GI irritations, and wherein the patient does not require urgent care.

Yet another embodiment provides a method of alleviating one or more symptoms of hangover, stomach flu or migraine in a patient in need thereof comprising inserting into a body cavity of the patient a suppository in a unit dosage form as described herein, wherein the symptoms include pain, nausea, and GI irritations, and wherein the patient does not require urgent care.

A further embodiment provides a method of alleviating one or more symptoms of hangover, stomach flu or migraine in a patient in need thereof comprising applying to the skin of the patient a first transdermal patch in a unit dosage form as described herein, wherein the symptoms include pain, nausea, and GI irritations, and wherein the patient does not require urgent care.

### DETAILED DESCRIPTION

Hangover symptoms often render the patients averse to the very measures that could alleviate their symptoms. For instance, nausea and gastro-intestinal irritations are often worsened when the patient ingests fluids and electrolytes with the intent to rehydrate and rebalance electrolytes. The effectiveness of the fluids and electrolytes is therefore limited if they are rapidly rejected by the body through vomiting and diarrhea, which in turn may cause additional loss of water and electrolytes. This inability to rehydrate and correct metabolic imbalance by ingestion further aggregates dehydration and metabolic imbalance, which are the main reasons for admitting the hangover patients into urgent care (i.e., intravenous fluids and/or intravenous medications).

Disclosed herein are various embodiments directed to pharmaceutical formulations, dosage forms and treatments that are capable of alleviating the symptoms of hangovers, stomach flu or migraine. Advantageously, sufficient relief is provided such that patients are capable of oral rehydrating and correcting any metabolic abnormalities through ingestion of appropriate fluids and electrolytes, thereby obviating the need for urgent care.

As used herein, “hangover” refers to a constellation of symptoms following alcohol consumption. The particular set of symptoms experienced and their intensity may vary from person to person. However, the symptoms that are shared by most of the hangover patients include nausea, gastrointestinal irritations, headaches and body/muscle aches. Often, dehydration and metabolic imbalance are among the underlying physiological causes.

Typically, considerable amounts of alcohol (e.g., >1.0 g/kg) need to be consumed to produce a hangover. However, the onset of hangover typically occurs after the blood alcohol concentration (BAC) begins to fall or returns to zero. Thus, hangovers usually begin within several hours after the end of alcohol consumption and can last from a few hours to up to about 48 hours. The severity of hangovers has been theorized to be related to the amount of cytokines produced and released from metabolizing alcohol.

Hangover is to be distinguished from “acute alcohol poisoning,” for which urgent care is necessary. Acute alcohol poisoning could occur during or after heavy consumption of alcohol, particularly within a short period of time. However, while the hangover symptoms generally precipitate from dehydration and metabolic imbalance; acute alcohol poisoning is the body's direct adverse reaction to alcohol intoxication. Acute alcohol poisoning therefore presents a different set of symptoms that include low body temperature, increased heart rate, lower blood pressure, continuous vomiting, irregular or slower breathing rates, confusion or stupor, potential seizures, cyanotic skin around lips and/or nail beds.

“Stomach flu” refers to any form of gastroenteritis (irritated and inflamed gastrointestinal tract) and presents one or more of the following symptoms: dehydration, vomiting, diarrhea, low grade fever, and aches throughout the body. Stomach flu may be caused by any number of pathogens (bacteria, virus and parasites) or chemical toxins (including alcohol). Viral infections (by e.g., adenoviruses, rotaviruses, calciviruses, astroviruses, and norovirus) are more common and highly contagious.

“Migraine” is understood to include a subset of headache characterized by unusual severity, unilateral, throbbing pain persisting for 4-72 hours and can further include one or more of the following symptoms: nausea, vomiting, sensitivity to light and/or sounds with or without a preceding “aura” and visual photophobia (e.g., visual disturbances).

The pharmaceutical composition, dosage forms and treatments according to various embodiments described herein are combination therapies of several therapeutically active agents. The combination is designed to alleviate the various symptoms of hangover, stomach flu or migraine by enhancing the absorption and efficacy of one or more individual agents, while managing or eliminating the potential side effects that may exacerbate the symptoms if any one of the active agents is taken alone.

### Pharmaceutical Formulation

One embodiment provides a pharmaceutical formulation comprising four categories of therapeutic agents, namely, an analgesic agent, a nonsteroidal anti-inflammatory agent, a gastric acid production suppressant, and an anti-nauseant, wherein the therapeutic agents are, respectively, in amounts sufficient to alleviate one or more symptoms of hangover, stomach flu or migraine.

The various therapeutic agents in each category are described in further detail below. It should be understood any one of the therapeutic agent of a given category may be combined with any one of the therapeutic agents of another category. Thus, all of the possible combinations of the therapeutic agents from each category are contemplated and are within the scope of this disclosure.

1. Analgesic Agent

An analgesic provides pain relief. As used herein, the analgesic agent is centrally acting and has no or little direct effect on inflammation. Its mode of action is typically by selectively blocking the cyclooxygenase (COX-2) in the central nervous system as opposed to the peripheral nervous system. Accordingly, the analgesic alleviates pain while having a minimal side effect to the gastrointestinal (GI) linings.

In a preferred embodiment, the analgesic is a non-opioid agent such as acetaminophen, chemically known as N-acetyl-p-aminophenol. Acetaminophen is the active ingredient in many analgesics (including those sold under the name TYLENOL). It is also known by its nonproprietary generic name paracetamol.

In another embodiment, the analgesic may be a mild opioid such as tramadol HCl, chemically known as (±)cis-2-[(dimethylamino)methyl]-1-(3-methoxyphenyl) cyclohexanol hydrochloride. Tramadol HCl is sold under the name ULTRAM®.

Other mild opioid analgesics may be codeine, hydrocodone, oxycodone, or a combination thereof.

2. Nonsteroidal Anti-Inflammatory Agent

A nonsteroidal anti-inflammatory agent or drug (NSAID) has a basic mode of action by inhibiting the pro-inflammatory enzyme cyclooxygenase (COX) in the central nervous system as well as the peripheral nervous systems, leading to a decrease in prostaglandin production. NSAIDs may be nonselective and thus nonspecifically inhibit both COX-1 and COX-2, or selective COX-2 inhibitors. NSAIDs thus are capable of reducing inflammation as well as pain.

Although NSAIDs may act on the central nervous system, they do not typically activate the opioid receptors and therefore produce pain relief by a different mechanism than opioid analgesic, as described herein.

NSAIDs are also different from acetaminophen, which has no direct anti-inflammatory effects.

NSAIDs encompass chemically diverse groups of agents, many but not all are carboxylic acid derivatives.

In various embodiments, the NSAID may be a propionic acid derivative such as ibuprofen (e.g., sold under the names ADVIL®, MOTRIN®, NUPRIN®), naproxen (e.g., sold under the name ALEVE®, NAPROSYN®), fenoprofen (e.g., sold under the name NALFON®), etodolac (e.g., sold under the name LODINE®), or flurbiprofen (e.g., sold under the name ANSAID®, FROBEN®).

In other embodiments, the NSAID may be an acidic acid derivative. Examples include, without limitation, heteroaryl acetic acids such as diclofenac; indole and indene acetic acids such as indomethacin (e.g., sold under the name INDOCIN®); naphthaleneacetic acid or derivative such as nabumatone (e.g., sold under the name RELAFEN®).

In other embodiments, the NSAID may be a sulfonamide derivative, which is typically a selectively COX-2 inhibitor. An example is celecoxib (sold under the name CELEBREX®).

In a further embodiment, the NSAID may be an oxicam such as meloxicam (e.g., sold under the name MOBIC®).

While both the analgesic and NSAID contribute to pain relief, the analgesic has only minimal effect on the GI lining. Moreover, in addition to directly producing pain relief, the NSAID also reduces the inflammation of the meningeal layer of the brain that causes headache. Thus, the dual action of the analgesic and the NSAID increase the potency of the pain relief while managing the potential irritation to the gastrointestinal lining.

3. Gastric Acid Production Suppressant

A gastric acid production suppressant protects the gastrointestinal (GI) lining by suppressing the secretin of gastric acid. Two modes of action may be at play, namely, by inhibiting proton pumps or by blocking histamine H2 receptors.

In certain embodiment, the gastric acid production suppressant is a proton pump inhibitor that shuts down the cell pumps that maintain the acidic environment in the GI track. Examples include, without limitation, omeprazole (e.g., sold under the name PRILOSEC®), pantoprazole (e.g., sold under the name PROTON IX®), and lansoprazole (e.g, sold under the name PREVACID®), esomeprazole (e.g., sold under the name NEXIUM®), and raberprazole (e.g, sold under the name ACIPHEX®).

In other embodiment, the gastric acid production suppressant is a H2 blocker that blocking signals generated by H2 histamine receptors on cells responsible for acid secretion. Examples include, without limitation, ranitidine (e.g., sold under the name ZANTAC®), famotidine (e.g., sold under the name PEPCID®), and cimetidine (e.g., sold under the name TAGAMET®).

The gastric acid production suppressant not only addresses the native symptoms of GI irritations brought on by hangover, stomach flu or migraine, but also protects the GI lining from being irritated by the NSAID, which is known to cause GI stress when taken alone. The protective effect allows the analgesic and NSAID be effectively absorbed through the GI lining to produce systemic pain relief.

The NSAID may be omitted for patients who might be allergic to NSAIDs.

4. Anti-Nauseant

An anti-nauseant is a drug effective against nausea and vomiting. The anti-nauseant typically acts on the central nervous system. The anti-nauseant may be an agent that is commonly used for treating nausea induced by motion sickness or side effects of chemotherapy against cancer or general anaesthetics.

In certain embodiments, the anti-nauseant is a serotonin 5-HT₃ receptor antagonist. Examples include, without limitation, dolasetron (e.g., sold under the name ANZEMET®), ondansetron (e.g., sold under the name ZOFRAN®), palonosetron (e.g., sold under the name ALOXI®).

In other embodiments, the anti-nauseant is a H1 receptor (antihistamine) antagonist. Examples include, without limitation, promethazine (e.g., sold under the name PHENERGAN®).

In further embodiments, the anti-nauseant is a dopamine (D2) receptor antagonist. Examples include, without limitation, prochlorperazine (e.g., sold under the name COMPAZINE®), droperidol (e.g., sold under the name INAPSINE®) and metoclopramide (e.g., sold under the name REGLAN®).

Once the nausea and vomiting are alleviated, the patient will be able to rehydrate and ingest electrolytes to correct any metabolic imbalance.

### Dosage Forms and Unit Dosage

Depending on the routes of administration, the pharmaceutical formulation described herein may be formulated into an oral dosage, a suppository, or a transdermal patch. The therapeutic agents are absorbed into the bloodstream through the stomach lining, rectal/vaginal mucosa, or skin, respectively.

A unit dosage form is a physically discrete unit, such as a tablet or capsule suitable as a unitary dosage for a patient. Each unit contains respective predetermined quantities of the therapeutic agents to produce the desired therapeutic effects. Different dosage forms may require different quantities of the therapeutic agents in each unit dosage.

In preferred embodiments, a single unit dosage form can bring relief such that rehydration through ingestion of liquids/electrolytes is possible. A second unit dose may be given 5-8 hours after the first dose, if needed.

1. Oral Dosage Form

Oral dosage forms are taken orally and release the therapeutic agents in the GI track. Oral dosage are available in many different forms, including tablet, soft gelatin capsule, hard gelatin capsule, suspension tablet, effervescent tablet, powder, effervescent powder, chewable tablet, suspension and the like.

In certain embodiment, the oral dosage, regardless of the specific form, may include a homogeneous mixture of an analgesic agent, a nonsteroidal anti-inflammatory agent, a gastric acid production suppressant, and an anti-nauseant, as described herein.

In other embodiment, the oral dosage is a solid tablet or capsule that has a layered structure formed by sequential coating of the therapeutic agents. The order of the coating determines the order of the release of the therapeutic agents once in the GI track. In particular, the layered structure may include a drug core having an analgesic and a NSAID. The drug core is coated or encapsulated by an intermediate layer having a gastric acid production suppressant. The intermediate layer is in turn further coated or encapsulated by an outer layer of an anti-nauseant. Once in the GI track, the layered structure releases the anti-nauseant first, to alleviate nausea and urge to vomit. The gastric acid production suppressant is then released to reduce the gastric acid secretion and GI irritation before the analgesic and the NSAID are released and absorbed.

In certain embodiments, the analgesic and NSAID are present in to a homogeneous mixture. In other embodiment, the analgesic and NSAID are present as two discrete layers or phases.

Besides the therapeutic agents, the oral dosage may further comprise inactive ingredients such as pregelatinized corn starch, modified starch (corn), hypromellose, lactose, magnesium stearate, microcrystalline cellulose, polyethylene glycol, polysorbate 80, sodium starch glycolate, titanium dioxide and carnauba wax. Additional inactive ingredients may be used as they are known in the art.

Thus, one embodiment provides a unit oral dosage form that comprises: 200-650 mg of an analgesic (e.g., acetaminophen), 20-800 mg of a nonsteroidal anti-inflammatory agent (e.g., naproxen, fenuprofen or flurbiprofen), 10-80 mg of a gastric acid production suppressant (e.g., famotidine) and 5-25 mg of an anti-nauseant (e.g., ondansetron).

A preferred embodiment provides a unit oral dosage form comprising 500 mg acetaminophen, 500 mg naproxen, 40 mg famotidine and 8 mg ondansetron.

Another preferred embodiment provides a unit oral dosage form comprising 500 mg acetaminophen, 800 mg fenuprofen, 40 mg famotidine and 8 mg ondansetron.

Yet another preferred embodiment provides a unit oral dosage form comprising 500 mg acetaminophen, 50-100 mg flurbiprofen, 40 mg famotidine and 8 mg ondansetron.

Yet another preferred embodiment provides a unit oral dosage form (e.g., a tablet or capsule) comprising 250 mg acetaminophen, 25 mg flurbiprofen, 10 mg famotidine and 5 mg ondansetron.

The unit dosage form may further comprise supplements such as vitamins (e.g., vitamin C, vitamin B12) and magnesium salt (e.g., magnesium stearate).

In certain embodiments, the unit dosage forms described herein may exclude the NSAID.

2. Suppository

Although oral dosage forms have the advantages of being easy to administer, they may not be practicable when the patient is unable to keep the oral dosage down. For example, in severe cases of hangover, stomach flu or migraine, the administration of the oral dosage may induce nausea and vomiting, whereby the oral dosage may be expelled before taking effect (in full or in part).

A suppository is a dosage form that can be inserted into a body cavity such as rectum and vagina, where the suppository dissolves or melts and the therapeutic agents are absorbed into the bloodstream through the mucosa of the cavities.

Thus, one embodiment provides a rectal or vaginal suppository thus comprises an analgesic agent, a nonsteroidal anti-inflammatory agent, a gastric acid production suppressant, and an anti-nauseant, as defined herein.

In a unit dosage form, a suppository may comprise: 200-650 mg of an analgesic (e.g., acetaminophen), 20-800 mg of a nonsteroidal anti-inflammatory agent (e.g., naproxen or fenuprofen), 10-80 mg of a gastric acid production suppressant (e.g., famotidine) and 5-25 mg of an anti-nauseant (e.g., ondansetron).

A preferred embodiment provides a unit suppository dosage form comprising 500 mg acetaminophen, 500 mg naproxen, 40 mg famotidine and 8 mg ondansetron.

Another preferred embodiment provides a unit suppository dosage form comprising 500 mg acetaminophen, 800 mg fenuprofen, 40 mg famotidine and 8 mg ondansetron.

Yet another preferred embodiment provides a unit suppository dosage form comprising 500 mg acetaminophen, 50-100 mg flurbiprofen, 40 mg famotidine and 8 mg ondansetron.

Yet another preferred embodiment provides a unit oral dosage form comprising 250 mg acetaminophen, 25 mg flurbiprofen, 10 mg famotidine and 5 mg ondansetron.

In certain embodiments, the unit dosage forms described herein may exclude the NSAID.

The combined therapeutic agents are typically comprised about 5-20%, 10-30%, 15-40%, 20-50%, 30-60%, or 40-70% of the total weight of the suppository, the remaining weight being inactive ingredients that form a matrix containing the therapeutic agents.

The inactive ingredients in a suppository are known to a skilled person in the art, and may include, for example, cocoa butter, oil (theobroma oil, hydrogenated cocoglyceride) or polyethylene glycol as the suppository bases. The therapeutic agents are dispersed in the suppository base. The coating may be a cellulosic film (e.g., hydroxypropyl methylcellulose film coating). The suppository may further include disintegrants (e.g., croscarmellose sodium and cross-linked povidone) to assist the disintegration.

3. Transdermal Patch

A transdermal dosage form (a patch) can also circumvent the oral administration route and is particularly useful in delivering a steady dose of the therapeutic agents through the skin and into the bloodstream. Advantageously, the therapeutic agents described herein are all small molecules that can efficiently penetrate the skin. Compared to the other dosage forms, which are relatively fast-acting, transdermal patches are capable of delivering the therapeutic agents in a sustainable manner over a specific period of time.

Suitable transdermal patches may take a number of configurations, including single layer or multi-layer structures. The therapeutic agents may be housed in a drug reservoir or a drug matrix. Upon contacting the skin, the therapeutic agents diffuse out of the drug reservoir or matrix, and through the skin.

One embodiment provides a transdermal patch comprising a backing, a drug matrix (e.g., in a thin film form) that includes the therapeutic agents dispersed in a matrix material (e.g., a propylene glycol, or a cellulosic material such as HPMC). The matrix material may be inherently tacky and functions as an adhesive, or a separate layer of adhesive (e.g., heat sensitive or pressure-sensitive) may be used.

In a unit dosage form, a transdermal patch may comprise a drug matrix that contains: 300-650 mg of an analgesic (e.g., acetaminophen), 100-800 mg of a nonsteroidal anti-inflammatory agent (e.g., naproxen or fenuprofen), 30-80 mg of a gastric acid production suppressant (e.g., famotidine) and 5-25 mg of an anti-nauseant (e.g., ondansetron).

A preferred embodiment provides a unit transdermal dosage form (e.g., a single patch) comprising 500 mg acetaminophen, 500 mg naproxen, 40 mg famotidine and 8 mg ondansetron.

Another preferred embodiment provides a unit transdermal dosage form (e.g., a single patch) comprising 500 mg acetaminophen, 800 mg fenuprofen, 40 mg famotidine and 8 mg ondansetron.

Another preferred embodiment provides a unit transdermal dosage form (e.g., a single patch) comprising 500 mg acetaminophen, 50 mg diclofenac, 40 mg famotidine, 8 mg ondansetron and 25 mg promethazine.

In certain embodiments, the unit dosage forms described herein may exclude the NSAID.

The combined therapeutic agents are typically comprised about 5-20%, 10-30%, 15-40%, 20-50%, 30-60%, or 40-70% of the total weight of the drug matrix, the remaining weight being inactive ingredients that form a matrix containing the therapeutic agents.

### Methods of Treatment

Various embodiments provide methods of alleviating one or more symptoms of hangover, stomach flu or migraine, wherein the symptoms include pain (e.g., headache and muscle pain), nausea, and GI irritations (e.g., vomiting and diarrhea). In particular, the methods make it possible for the patient to overcome the symptoms and to be in a position to ingest fluids and electrolytes without requiring urgent care.

The symptoms are believed to be the manifestations of a number of physiological stress factors such as dehydration, metabolite imbalance and/or inflammation. The severity and duration of the symptoms of hangover, stomach flu or migraine can vary from patient to patient and are largely self-reporting.

“Alleviating” refers to a lessening in severity, and/or a shortening in duration of a given symptom after administering the pharmaceutical formulation or dosage form, when compared to the same symptom that goes untreated.

“Patient” refers to a human subject who has consumed a sufficient quantity of alcohol to develop one or more symptoms of hangover after the alcohol consumption ended. The patient experiences dehydration and metabolic imbalance. In certain embodiments, the patient has a blood alcohol concentration of zero.

“Patient” could also be a human subject inflicted by any form of gastroenteritis and develops one or symptoms including pain (e.g., headache and muscle pain), nausea, and GI irritations (e.g., vomiting and diarrhea), similar to those of hangover.

“Patient” could also be a human subject inflicted by migraine and develops one or symptoms including pain (e.g., headache and muscle pain), nausea, and GI irritations (e.g., vomiting and diarrhea), similar to those of hangover.

“Urgent care” is a necessary treatment at a stage of hangover, stomach flu, or migraine whereby the patient is unable to ingest fluids or electrolytes due to the one or more symptoms; and as a result, the dehydration and electrolyte imbalance become sufficient severe that the patient requires intravenous fluids and medications. Urgent care therefore marks an acute stage for hangover, stomach flu, or migraine. A physician or qualified urgent care personnel may determine whether a patient has reached the acute stage.

The methods described herein are therefore directed to alleviating symptoms and allowing the patient to rehydrate and correct metabolic imbalance before ever reaching the acute stage of the hangover, stomach flu or migraine.

One embodiment provides a method of alleviating one or more symptoms of hangover, stomach flu or migraine in a patient in need thereof comprising orally administering to the patient a single unit dosage form including: 200-650 mg of an analgesic (e.g., acetaminophen), 20-800 mg of a nonsteroidal anti-inflammatory agent (e.g., naproxen or fenuprofen), 10-80 mg of a gastric acid production suppressant (e.g., famotidine) and 5-25 mg of an anti-nauseant (e.g., ondansetron), wherein the symptoms include pain (e.g., headache and muscle pain), nausea, and GI irritations (e.g., vomiting and diarrhea), and wherein the patient does not require urgent care.

Advantageously, the single oral dosage delivers a combination of therapeutic agents that target the symptoms while managing or minimizing potential adverse effects that may further aggregate the symptoms had one or more therapeutic agents been taken alone. It is easy to administer and to achieve a high level of patient compliance.

In various embodiments, the single unit dosage form is a tablet or a capsule that combines at least one therapeutic agent from each of the four categories, namely, an analgesic, a NSAID, a gastric acid production suppressant and an anti-nauseant. In a preferred embodiment, the tablet or capsule has a layered structure that comprises discrete layers of each one of the therapeutic agents. Alternatively, the analgesic and the NSAID may together form a homogeneous drug core, with only the gastric acid production suppressant and the anti-nauseant in discrete layers. This configuration thus allows for a sequential release of the therapeutic agent and provides certain temporal control over the alleviating events. For example, the method targets nausea first, followed by releasing the gastric acid production suppressant before the analgesic and NSAID are released and absorbed. In this manner, the GI track is given a chance to prepare or protect itself before receiving the analgesic and NSAID.

Another embodiment provides a method of alleviating one or more symptoms of hangover, stomach flu or migraine in a patient in need thereof comprising orally administering to the patient, sequentially, (1) a single unit dosage form of an anti-nauseant, followed by (2) a single unit dosage form of an gastric acid production suppressant, followed by (3) a single unit dosage form of an analgesic, and followed by (4) a single unit dosage form of a nonsteroidal anti-inflammatory agent, wherein the symptoms include pain (e.g., headache and muscle pain), nausea, and GI irritations (e.g., vomiting and diarrhea), and wherein the patient does not require urgent care.

In other embodiments, the order of the (3) and (4) may be reversed.

The time interval between each step may be determined by the degree of alleviation and may be in the range of seconds to minutes. For instance, the anti-nauseant and the gastric acid production suppressant may be taken a few seconds apart. Several minutes (e.g., 2 minutes) thereafter, acetaminophen may be taken, followed by another 2 minutes interval before the NSAID is taken. The sequential oral administration of individual single dosage forms of the therapeutically agents could provide a pharmacokinetic profile similar to that of the single unit dosage form that combines all of the therapeutic agents in a layered structure.

In more specific embodiment, the method comprises orally and administering (1) a single unit dosage of ondansetron (5-8 mg), followed by (2) a single unit dosage of famotidine (10-40 mg), followed by (3) a single unit dosage of acetaminophen (250-500 mg), followed by (4) a single unit dosage of naproxen or flurbiprofen (20-500 mg). In other embodiments, the order of the (3) and (4) may be reversed.

Another embodiment provides a method of alleviating one or more symptoms of hangover, stomach flu or migraine in a patient in need thereof comprising inserting into a body cavity of the patient a suppository in a unit dosage form that includes: 200-650 mg of an analgesic (e.g., acetaminophen), 20-800 mg of a nonsteroidal anti-inflammatory agent (e.g., naproxen or fenuprofen), 10-80 mg of a gastric acid production suppressant (e.g., famotidine) and 5-25 mg of an anti-nauseant (e.g., ondansetron), wherein the symptoms include pain (e.g., headache and muscle pain), nausea, and GI irritations (e.g., vomiting and diarrhea), and wherein the patient does not require urgent care.

In various embodiments, the body cavity is rectum or vagina.

Yet another embodiment provides a method of alleviating one or more symptoms of hangover, stomach flu or migraine in a patient in need thereof comprising applying to the skin of the patient a first transdermal patch in a unit dosage form that includes: 200-650 mg of an analgesic (e.g., acetaminophen), 20-800 mg of a nonsteroidal anti-inflammatory agent (e.g., naproxen or fenuprofen), 10-80 mg of a gastric acid production suppressant (e.g., famotidine) and 5-25 mg of an anti-nauseant (e.g., ondansetron), wherein the symptoms include pain (e.g., headache and muscle pain), nausea, and GI irritations (e.g., vomiting and diarrhea), and wherein the patient does not require urgent care.

In various embodiments, the method further comprises removing the first patch after a period of 5-8 hours, and applying a second transdermal patch in the unit dosage form.

### Examples

### Formulation 1

Capsules having the following ingredients in powder form were prepared.

| Ingredients | Weight (mg) |
|---|---|
| Acetaminophen | 250 |
| Famotidine | 10 |
| Flurbiprofen | 25 |
| Ondansetron HCl | 5 |
| Lactose monohydrate | 33 |

Additional pharmaceutically acceptable fillers or binders (e.g., starch) were added to bring the total weight of the ingredients in each capsule to 1000 mg.

### Formulation 2

Capsules having the following ingredients in powder form were prepared, which included supplements of magnesium salt and vitamin C (ascorbic acid).

| Ingredients | Weight (mg) |
|---|---|
| Acetaminophen | 250 |
| Famotidine | 10 |
| Flurbiprofen | 25 |
| Ondansetron HCl | 5 |
| Lactose monohydrate | 10 |
| Magnesium Stearate | 10 |
| Ascorbic acid | 10 |

Additional pharmaceutically acceptable fillers or binders (e.g., starch) may be added to bring the total weight of the ingredients in each capsule to 1000 mg.

### Formulation 3

Capsules having the following ingredients in powder form were prepared, which further included supplements such as vitamin B12 (riboflavin).

| Ingredients | Weight (mg) |
|---|---|
| Acetaminophen | 250 |
| Famotidine | 10 |
| Flurbiprofen | 25 |
| Ondansetron HCl | 5 |
| Lactose monohydrate | 33 |
| Riboflavin | 1 |

Additional pharmaceutically acceptable fillers or binders (e.g., starch) may be added to bring the total weight of the ingredients in each capsule to 1000 mg.

From the foregoing it will be appreciated that, although specific embodiments of the invention have been described herein for purposes of illustration, various modifications may be made without deviating from the spirit and scope of the invention.

1. A pharmaceutical composition comprising:
an analgesic agent,
a nonsteroidal anti-inflammatory agent,
a gastric acid production suppressant, and
an anti-nauseant,
wherein the analgesic agent, the nonsteroidal anti-inflammatory agent, the gastric acid production suppressant, and the anti-nauseant are, respectively, in sufficient amounts to alleviate one or more symptoms of hangover, stomach flu or migraine.

2. The pharmaceutical composition of claim 1 wherein the analgesic agent is acetaminophen, tramadol HCl, codeine, hydrocodone, oxycodone, or a combination thereof.

3. The pharmaceutical composition of claim 1 wherein the nonsteroidal anti-inflammatory agent is ibuprofen, naproxen, fenoprofen, etodolac, diclofenac, indomethacin, nabumatone, celecoxib, flurbiprofen, meloxicam, or a combination thereof.

4. The pharmaceutical composition of claim 1 wherein the gastric acid production suppressant is omeprazole, pantoprazole, lansoprazole, esomeprazole, raberprazole, ranitidine, famotidine, cimetidine, or a combination thereof.

5. The pharmaceutical composition of claim 1 wherein the anti-nauseant is dolasetron, ondansetron, palonosetron, promethazine, prochlorperazine, droperidol, metoclopramide, or a combination thereof.

6. The pharmaceutical composition of claim 1 in an oral dosage form, a suppository or a transdermal patch.

7. A unit dosage form for alleviating one or more symptoms of hangover, stomach flu or migraine, the unit dosage form comprising:
200-650 mg of an analgesic agent;
20-800 mg of a nonsteroidal anti-inflammatory agent;
10-80 mg of a gastric acid production suppressant; and
5-25 mg of an anti-nauseant.

8. The unit dosage form of claim 7 wherein the analgesic agent is acetaminophen, tramadol HCl, codeine, hydrocodone, oxycodone, or a combination thereof.

9. The unit dosage form of claim 7 wherein the nonsteroidal anti-inflammatory agent is ibuprofen, naproxen, fenoprofen, etodolac, diclofenac, indomethacin, nabumatone, celecoxib, flurbiprofen, meloxicam, or a combination thereof.

10. The unit dosage form of claim 7 wherein the gastric acid production suppressant is omeprazole, pantoprazole, lansoprazole, esomeprazole, raberprazole, ranitidine, famotidine, cimetidine, or a combination thereof.

11. The unit dosage form of claim 7 wherein an anti-nauseant is dolasetron, ondansetron, palonosetron, promethazine, prochlorperazine, droperidol, metoclopramide, or a combination thereof.

12. The unit dosage form of claim 7 in an oral dosage form, a suppository or a transdermal patch.

13. The unit dosage form of claim 7 being a capsule or tablet comprising 250 mg acetaminophen, 25 mg flurbiprofen, 10 mg famotidine, and 5 mg ondansetron.

14. The unit dosage form of claim 13 in a layered structure comprising:
a drug core including the analgesic agent and the nonsteroidal anti-inflammatory agent;
an intermediate layer encapsulates the drug core, the intermediate layer including the gastric acid production suppressant; and
an outer layer encapsulating the intermediate layer, the outer layer including the anti-nauseant.

15. A method of alleviating one or more symptoms of hangover, stomach flu or migraine in a patient in need thereof comprising: orally administering to the patient one or more single unit dosage forms of claim 7, wherein the symptoms include pain, nausea, and GI irritations, and wherein the patient does not require urgent care.

16. A method of alleviating one or more symptoms of hangover, or stomach flu or migraine in a patient in need thereof comprising orally administering to the patient, sequentially,
(1) a single unit dosage form of an anti-nauseant, followed by
(2) a single unit dosage form of an gastric acid production suppressant, followed by
(3) a single unit dosage form of an analgesic, and followed by
(4) a single unit dosage form of a nonsteroidal anti-inflammatory agent, wherein the symptoms include pain, nausea, and GI irritations, and wherein the patient does not require urgent care.

17. The method of claim 16 comprising orally administering, sequentially,
(1) a single unit dosage of ondansetron (5-8 mg), followed by
(2) a single unit dosage of famotidine (10-40 mg), followed by
(3) a single unit dosage of acetaminophen (250-500 mg), and followed by
(4) a single unit dosage of naproxen or flurbiprofen (20-500 mg).

18. The method of claim 16 wherein the step of (3) and (4) are reversed.

19. A method of alleviating one or more symptoms of hangover, stomach flu or migraine in a patient in need thereof comprising inserting into a body cavity of the patient a suppository in a unit dosage form of claim 7, wherein the symptoms include pain, nausea, and GI irritations, and wherein the patient does not require urgent care.

20. A method of alleviating one or more symptoms of hangover, stomach flu or migraine in a patient in need thereof comprising applying to the skin of the patient a first transdermal patch in a unit dosage form of claim 7, wherein the symptoms include pain, nausea, and GI irritations, and wherein the patient does not require urgent care.

21. (canceled)
